(19) United States
(12) Patent Application Publication
(10) Pub. No.: US 2008/0107569 A1
(43) Pub. Date: May 8, 2008

(54) DETECTING NITRIC OXIDE

(76) Inventors: George B. Stefano, Melville, NY (US); Kirk J. Mantione, Patchogue, NY (US)

Correspondence Address: FISH & RICHARDSON P.C., PO BOX 1022, MINNEAPOLIS, MN, 55440-1022, US

(21) Appl. No.: 11/855,817

(22) Filed: Sep. 14, 2007

Related U.S. Application Data
(60) Provisional application No. 60/825,681, filed on Sep. 14, 2006.

(51) Int. Cl. G01N 33/497

(52) U.S. Cl. 422/84; 73/23.3

(57) ABSTRACT

This document provides methods and materials that can be used to measure NO. For example, NO sensing devices, methods for making NO sensing devices, and methods for using NO sensing devices to measure NO in, for example, exhaled human breath are provided.

### CROSS-REFERENCE TO RELATED APPLICATIONS

This application claims priority to U.S. Provisional Application No. 60/825,681, filed Sep. 14, 2006, which is incorporated by reference in its entirety.

### BACKGROUND

1. Technical Field

This document relates to methods and materials involved in detecting nitric oxide. For example, this document relates to methods and materials involved in measuring nitric oxide in exhaled breath.

2. Background Information

Nitric oxide (NO) is a gaseous signal molecule that can originate from cells such as neural, immune, and vascular cells, which express constitutive nitric oxide synthase (cNOS) or inducible NOS (Stefano et al., Progress in Neurobiology, 60:531-544 (2000)). NO is a free radical, which makes it very reactive and unstable. In air, NO can quickly react with oxygen to form nitrogen dioxide.

NO can be measured using a chemiluminescent reaction involving ozone. For example, a sample containing NO can be mixed with a large quantity of ozone. The NO can react with the ozone to produce oxygen and nitrogen dioxide. This reaction can produce light (e.g., chemiluminescence), which can be measured using a photodetector. See, formula 1. The amount of light produced can be proportional to the amount of NO in the sample.
NO+O₃→NO₂+O₂+light (Formula 1)

### SUMMARY

This document provides methods and materials that can be used to measure NO. For example, this document provides NO sensing masks, methods for making NO sensing masks, and methods for using NO sensing masks to measure NO in, for example, exhaled human breath. Using the methods and materials provided herein to measure NO can allow clinicians and researchers to determine NO levels in a quick, convenient, and sensitive manner. For example, the methods and materials provided herein can be used to measure NO levels in exhaled human breath in real time while the human is awake and active. The sensitivity of NO measurements using the methods and materials provided herein can be within the 1 part per billion (ppb) range.

In general, one aspect of this document features a device for sensing nitric oxide in exhaled breath. The device comprises, or consists essentially of, a mask portion configured to cover the nose and mouth of a mammal, thereby forming a sensing chamber when applied to the mammal, and a nitric oxide probe (e.g., amperometric probe) for sensing nitric oxide within the sensing chamber. The mask portion can comprise cloth or paper. The mammal can be a human. The device can comprise a connector for positioning the mask portion over the nose and mouth of a mammal. The connector can comprise an elastic cord. The mask portion can define an opening for the nitric oxide probe. The nitric oxide probe can be an amperometric, non-chemiluminescence probe. The mask portion can comprise moisture. The device can comprise a fluid reservoir capable of providing moisture to a surface of the mask portion. The surface can be an inner surface of the mask portion. The mask portion can comprise a pleat.

In another aspect, this document features a method for making a device for sensing nitric oxide in exhaled breath. The method comprises, or consists essentially of:

(a) obtaining a nitric oxide probe (e.g., amperometric probe) for sensing nitric oxide;

(b) obtaining a mask portion configured to cover the nose and mouth of a mammal, thereby forming a sensing chamber when applied to the mammal, and

(c) adding the probe to the mask portion such that the probe is capable of sensing NO within the sensing chamber. The mask portion can comprise cloth or paper. The mammal can be a human. The method can comprise adding, to the mask portion, a connector for positioning the mask portion over the nose and mouth of a mammal. The connector can comprise an elastic cord. The mask portion can define an opening for the nitric oxide probe. The nitric oxide probe can be an amperometric, non-chemiluminescence probe. The mask portion can comprise moisture. The method can comprise adding, to the mask portion, a fluid reservoir capable of providing moisture to a surface of the mask portion. The surface can be an inner surface of the mask portion. The mask portion can comprise a pleat.

In another aspect, this document features a method for sensing nitric oxide in exhaled breath. The method comprises, or consists essentially of:

(a) obtaining a device comprises a mask portion configured to cover the nose and mouth of a mammal, thereby forming a sensing chamber when applied to the mammal, and a nitric oxide probe (e.g., amperometric probe) for sensing nitric oxide within the sensing chamber;

(b) applying the device to the face of the mammal, thereby forming the sensing chamber; and

(c) sensing exhaled NO within the sensing chamber via the probe. The mask portion can comprise cloth or paper. The mammal can be a human. The device can comprise a connector for positioning the mask portion over the nose and mouth of a mammal. The connector can comprise an elastic cord. The mask portion can define an opening for the nitric oxide probe. The nitric oxide probe can be an amperometric, non-chemiluminescence probe. The mask portion can comprise moisture. The device can comprise a fluid reservoir capable of providing moisture to a surface of the mask portion. The surface can be an inner surface of the mask portion. The mask portion can comprise a pleat. The method can comprise applying moisture to a surface of the mask portion before or after the applying step (b).

In another aspect, this document features a device for sensing nitric oxide in exhaled breath of a mammal. The device comprises a mouthpiece portion, an extender portion, and a nitric oxide sensing chamber portion configured to allow exhaled breath to travel from the mouthpiece portion to the nitric oxide sensing chamber portion by traveling through the extender portion, wherein the device comprises a flow restrictor within the mouthpiece portion, the extender portion, or the nitric oxide sensing chamber portion, and wherein the device comprises a nitric oxide probe (e.g., amperometric probe) for sensing nitric oxide within the nitric oxide sensing chamber portion. The mammal can be a human. The nitric oxide probe can be an amperometric, non-chemiluminescence probe. The device can comprise moisture. The device can comprise a fluid reservoir capable of providing moisture to a inner surface of the device.

In another aspect, this document features a device for sensing nitric oxide in exhaled breath. The device comprises a portion configured to cover the nose or mouth of a mammal, thereby forming a sensing chamber when applied to the mammal, and an amperometric, non-chemiluminescence probe for sensing nitric oxide within the sensing chamber. The portion can be a mask portion comprising cloth or paper. The mammal can be a human. The device can comprise a connector for positioning the portion over the nose and mouth of a mammal. The connector can comprise an elastic cord.

In another aspect, this document features a method for sensing nitric oxide in exhaled breath. The method comprises: (a) obtaining a device comprises a portion configured to cover the nose or mouth of a mammal, thereby forming a sensing chamber when applied to the mammal, and an amperometric, non-chemiluminescence probe for sensing nitric oxide within the sensing chamber; (b) applying the device to the mammal, thereby forming the sensing chamber; and (c) sensing exhaled NO within the sensing chamber via the probe. The portion can comprise a mask portion comprising cloth or paper. The mammal can be a human. The method can comprise applying moisture to a surface of the device. The device can comprise a mouthpiece portion, an extender portion, and a nitric oxide sensing chamber portion configured to allow exhaled breath to travel from the mouthpiece portion to the nitric oxide sensing chamber portion by traveling through the extender portion, wherein the device comprises a flow restrictor within the mouthpiece portion, the extender portion, or the nitric oxide sensing chamber portion.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a side view of one example of an NO sensing mask.

FIG. 2 is a graph plotting current (pA) versus the concentration of NO in parts per million measured using samples containing zero, 10, and 51 parts per million (ppm) of NO.

FIG. 3 contains two graphs plotting NO concentration (parts per billion (ppb)) versus time (seconds) for exhaled breath from two humans (one graph for each human) while in a normal non-moving state after just sitting down (e.g., sitting).

FIG. 4 is a graph plotting NO in ambient air versus time.

FIG. 5 contains two graphs plotting NO concentration (ppb) versus time (seconds). The top graph is for pure CO₂, while the bottom graph is for 100% humidity air, demonstrating that these potential influences are not substantial.

FIG. 6 contains two graphs plotting NO concentration (ppb) versus time (seconds) for exhaled breath from two humans (one graph for each human) pre and post exercise.

FIG. 7 is a side view of an example of an NO sensing device.

FIG. 8 is a graph plotting current (pA) versus the concentration of NO in ppb measured using samples containing zero, 52 ppb, 10 ppm, and 51 ppm of NO.

### DETAILED DESCRIPTION

This document provides methods and materials related to the sensing NO. For example, this document provides NO sensing devices, methods for making NO sensing devices, and methods for sensing NO.

In general, an NO sensing device provided herein can be configured to create a sensing chamber that is formed between an inner surface of a mask and the user's face. The user can be any type of mammal including, without limitation, a human, dog, cat, cow, horse, pig, sheep, or monkey. This sensing chamber can provide an environment for sensing NO exhaled by the user. In some cases, an NO sensing device provided herein can contain an electrode capable of sensing NO within a sensing chamber. For example, an NO sensing device provided herein can contain a mask defining an opening to a sensing chamber that contains an electrode designed to sense NO within the sensing chamber.

An NO sensing device provided herein can be easily adjustable and can provide a comfortable fit. For example, an NO sensing device can have an elastic strap designed to hold the mask in position on a user's face. In some cases, the NO sensing devices provided herein can provide a barrier about the nose and mouth of a user and at least a portion of the user's cheeks, jaw, and chin. An NO sensing mask provided herein can contain one or more layers of filter media or barrier material designed to filter the passage of aerosols, fluids, and/or particulate matter. In some embodiments, an NO sensing device provided herein can be constructed so that the material of the mask portion can be moistened with, for example, water or a saline solution prior to sensing NO within the sensing chamber. In some cases, moisture present in or on the mask portion can create a moist environment within the sensing chamber. This moist environment can aid in the detection and accurate measurement of NO within the sensing chamber.

In some cases, an NO sensing device provided herein can be configured as a mouthpiece (e.g., a handheld mouthpiece). For example, a hollow structure (e.g., a tubular structure) can be used to form a sensing chamber. In such cases, a user can breathe into one end of the hollow structure, and a probe positioned within the hollow structure can measure the level of NO within the hollow structure or a portion of the hollow structure. The exhaled breath can exit the hollow structure after passing the probe. In some cases, a portion of the exhaled breath can exit the hollow structure before passing the probe provided that sufficient air flow exists around the probe. In some cases, a device provided herein can be configured to have one or more flow restrictors (e.g., a dynamic flow restrictor). Such flow restrictors can be designed to allow exhaled breath to enter a separate chamber containing a probe. In some cases, flow restrictors can be used such that exhaled breath exits the device, after sensing NO with a probe, without being returned to the user.

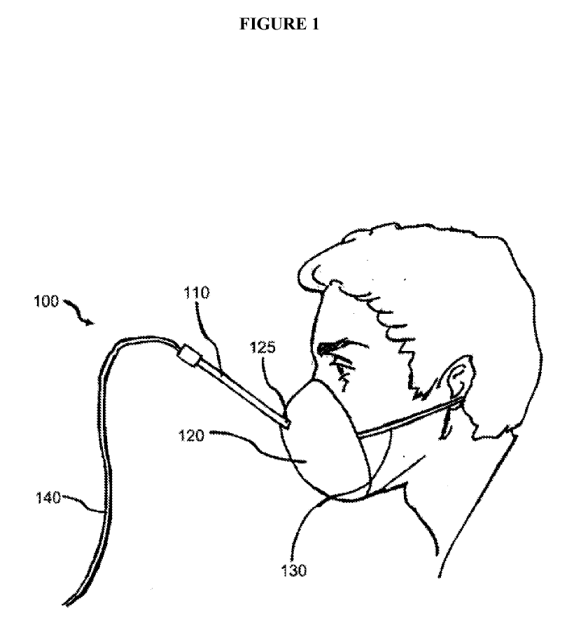

With reference to FIG. 1, device 100 can contain mask portion 120, which can be positioned over a portion of a user's face such as the user's nose, mouth, and portions of the user's cheeks, jaw, and chin. Mask portion 120 can substantially cover the user's nose and mouth, or either separately. In some cases, a nose plug can be used to restrict breathing through the nose. For example, a device designed to engage a user's mouth can be used in combination with a nose plug. As shown in FIG. 1, mask portion 120 can generally lack pleats. For example, mask portion 120 can be cone-shaped, duck bill-shaped, or a similar single fold, and/or non-collapsible-shaped. These types of mask portions can provide “off-the-face” benefits such as being easy to stack, package, store, and ship. Cone-shaped, duck bill-shaped, and non-collapsible shaped “off-the-face”-style masks can provide, to some users, a larger breathing chamber as compared to soft, pleated masks which may contact more of the user's face. Examples of generally cone-shaped masks are disclosed in U.S. Pat. Nos. 4,536,440 and 4,729,371. Many cone-style face masks are known and commercially available. An example of a generally duck bill-shaped mask is disclosed in U.S. Pat. No. 4,606,341. Examples of generally non-collapsible shaped masks are disclosed in U.S. Pat. Nos. 6,055,982 and 6,173,712. In some cases, mask portion 120 can be pleated. Examples of pleated masks are disclosed in U.S. Pat. Nos. 4,635,628; 4,969,457; and 4,920,960. Many pleated masks are known and commercially available.

Mask portion 120 can made from any type of material including, without limitation, paper (e.g., filter paper) or cloth (e.g., silk, cotton, polyester fabric, nylon, or combinations thereof). In some cases, mask portion 120 can include barrier material. The barrier material can be positioned so that aerosols, fluids, and/or particulate matter contacting device 100 from the outside will be filtered. The barrier material can be positioned on any inner or outer surface of the mask, or in any layer intermediate to an inner or outer surface. The barrier material can include filtration media, which can be, for example, melt-blown polypropylene or polyester. The filtration media can be provided to reduce the passage of, for example, airborne bacteria in either direction. In addition, the barrier material can include an inner layer that contacts the face of the user. Such an inner layer can be constructed of a light weight, highly porous, softened, non-irritating, non-woven fabric. Such an inner layer can be designed to provide a comfortable surface for contact with the face of the user. One barrier material or more than one barrier material can be used. Further description of the construction and operation of such barrier material is provided elsewhere (e.g., U.S. Pat. Nos. 3,929,135 and 6,055,982). Exemplary barrier materials include, but are not limited to, those described elsewhere (e.g., U.S. Pat. Nos. 4,635,628; 4,969,457; and 4,920,960).

As described herein, an NO sensing device can be constructed so that the material of the mask portion can be moistened with, for example, water, a saline solution, or a water gel composite prior to sensing NO within the sensing chamber. In some cases, an NO sensing device provided herein can contain a fluid reservoir capable of holding fluid such as water. Such a fluid reservoir can be configured to deliver fluid to a surface of the mask portion. For example, a fluid reservoir can be configured to deliver a fine mist to the inner surface of a mask portion so that a moist environment is created within the sensing chamber. Any type of dispensing unit can be used to deliver fluid to the sensing unit. For example, a push bulb spray unit can be actuated by a user to deliver a fine mist to the sensing chamber. In some cases, a user can use a spray bottle to moisten an inner surface of a mask portion prior to applying the device to the user's face.

In some embodiments, a top edge of a mask portion can include an elongated malleable member. Such a malleable member can be configured to allow the top edge of a mask portion to fit the contours of the nose and upper cheeks of the user closely. The malleable member can be constructed from a metal strip with a rectangular cross-section, but can form any suitable configuration, and also can be a moldable or a malleable metal or alloy, plastic, or any combination thereof.

Device 100 can contain connector 130. Connector 130 can be configured to position mask portion 120 to a user's face. Connector 130 can be a pair of ties that can be fastened together in a traditional manner to the user's face via tying the ties in a bow, knot, and so forth, at the back of the user's head. The ties can be un-fastened to release the mask portion from the user's face. In some cases, connector 130 can be a cord, a strap, a string, and/or a ribbon constructed from an elastomeric and/or non-elastomeric material. For example, connector 130 can be constructed of rubber, elastic covered yarn, an elastomeric material wrapped with nylon or polyester, and so forth.

Mask portion 120 of device 100 can define opening 125. Opening 125 can be configured so that probe 110 can sense NO within a sensing chamber. Opening 125 can be any size or shape. Typically, opening 125 matches the size and shape of probe 110 so that a snug fit is formed between mask portion 120 and probe 110. In some cases, an adapter can be used as an interface between mask portion 120 and probe 110. Such an adapter can be constructed from a material different from the material used to construct the mask portion. In some cases, an adaptor can be a circular shaped sleeve that provides extra reinforcement for the mask portion in the region surrounding opening 125. Probe 110 can access the sensing chamber via opening 125. Any type of probe (e.g., amperometric probe) can be used provided that it is capable of sensing NO. Examples of probes that can be used include, without limitation, those produced or sold by Diamond General (Ann Arbor, Mich.), World Precision Instruments (Sarasota, Fla.), Innovative Instruments, Inc. (Tampa, Fla.), Inter Medical Co., Ltd. (Nagoya, Japan), and TSI Incorporated (Shoreview, Minn.). In some cases, a selective amperometric combination electrode or differential electrode can be used as a probe to sense NO as described herein. In some cases, a non-chemiluminescence, amperometric probe capable of sensing NO can be used as described herein. For example, an electrochemical probe capable of sensing NO can be used as described herein. In some cases, probe 110 can be designed to sense pH, moisture, and temperature within a sensing chamber.

Probe 110 can be wired via wire 140 to an analyzer capable of receiving NO sensing data from the probe. Such an analyzer also can provide output about NO levels detected within a sensing chamber. Examples of analyzers include, without limitation, those produced or sold by ESA Biosciences, Inc. (Chelmsford, Mass.), Innovative Instruments, Inc. (Tampa, Fla.), Inter Medical Co., Ltd. (Nagoya, Japan), Diamond General (Ann Arbor, Mich.), TSI Incorporated (Shoreview, Minn.), EDAQ (New South Wales, Australia), World Precision Instruments (Sarasota, Fla.). In some cases, a WPI Apollo 4000 analyzer, a DUO18 analyzer, or ESA Biostat can be used. In some cases, probe 110 can be wireless such that NO sensing data is sent from probe 110 to an analyzer in a wireless manner.

In some cases, a device provided herein can be configured to have a flow restrictor (e.g., a dynamic flow restrictor) designed to allow exhaled breath to enter a separate chamber containing a probe. In such cases, the exhaled breath can exit the device, after sensing NO with a probe, without being returned to the user.

In a manner of use, device 100 can be put on by the user pulling the mask portion 120 over the user's nose and mouth while positioning connector 130 around the back of the user's head. The malleable member, if included, can be positioned across the user's nose and the top edge of mask portion 120. In some cases, the outer side, inner side, or both the outer and inner sides of the mask portion can be moistened with water via a spray bottle before or after being put on the user's face. Once the NO sensing device is in position, the user can breath normally or under various conditions (e.g., while walking or running on a tread-mill, while mediating (e.g., relaxing), or while sleeping) for a pre-selected time period (e.g., 0.5, 1, 2, 5, 10, 20, or 30 minutes). In some cases, NO can be measured in users having a particular disease or condition. For example, NO can be measured in a group of asthma patients. The probe can be used to sense NO within the sensing chamber in either a continuous mode or at pre-set intervals (e.g., once every 10, 30, or 60 seconds). Prior to making a NO measurement, the probe can be calibrated. For example, when sensing exhaled NO, an initial three point calibration can be performed followed by daily two point calibrations (e.g., zero and a point in the expected range). See, e.g., An official statement of the American Thoracic Society adopted by the ATS Board of Directors, July 1999 (Am. J. Respir. Crit. Care Med., 160(6):2104-17 (1999)).

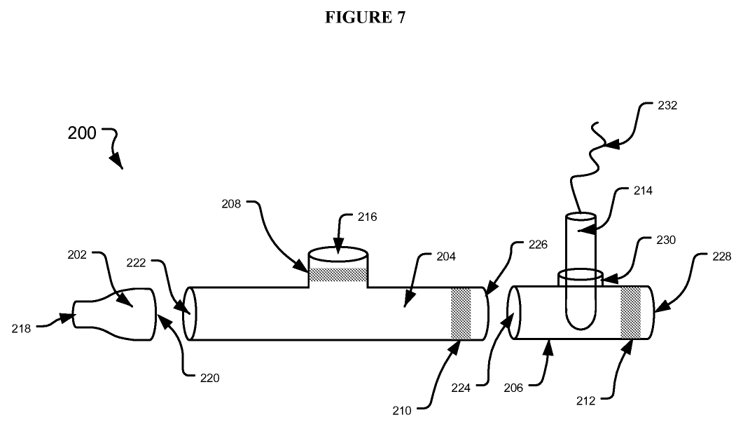

With reference to FIG. 7, device 200 can contain mouthpiece portion 202, extender portion 204, and sensing chamber portion 206. Mouthpiece portion 202 can be designed to engage a user's mouth, a user's nostril, or both. In some cases, a nose plug can be used to restrict breathing through the nose. For example, device 200 can be used in combination with a nose plug. As shown in FIG. 7, mouthpiece portion 202 can be a separate, disposable unit. In some cases, mouthpiece portion 202 can be constructed as an integral unit together with extender portion 204, sensing chamber portion 206, or both extender portion 204 and sensing chamber portion 206. Mouthpiece portion 202 can have inlet port 218, which can receive exhaled breath from a user. Exhaled breath can exit mouthpiece portion 202 through outlet port 220 and enter extender portion 204 via inlet port 222. Extender portion 204 can be configured to control flow rate or direction of exhaled breath within device 200. For example, extender portion 204 can contain one or more exit ports (e.g., exit port 216). Exit port 216 can allow a portion of exhaled breath to exit device 200 without coming into contact with NO sensing probe 214. Such an exit port can contain an air flow restrictor 208. Air flow restrictor 208 can be designed to allow exhaled breath to exit through exit port 216 in a manner that is restricted as compared to an exit port lacking an air flow restrictor. An air flow restrictor can be made from any material including, without limitation, polyethylene, polyvinylchloride, or latex. As shown in FIG. 7, extender portion 204 can have outlet port 224. Exhaled breath can exit extender portion 204 through outlet port 224 and enter sensing chamber portion 206 via inlet port 226. Extender portion 204 can be a separate, disposable unit. In some cases, extender portion 204 can be constructed as an integral unit together with mouthpiece portion 202, sensing chamber portion 206, or both mouthpiece portion 202 and sensing chamber portion 206. Outlet port 224 of extender portion 204 can be configured to contain air flow restrictor 210. Air flow restrictor 210 can be designed to allow exhaled breath to exit through outlet port 224 in a manner that is restricted as compared to an outlet port lacking an air flow restrictor.

With further reference to FIG. 7, sensing chamber portion 206 can have outlet port 228. Exhaled breath can exit sensing chamber portion 206 through outlet port 228 such that it is not returned to the user. Sensing chamber portion 206 can be a separate, disposable unit. In some cases, sensing chamber portion 206 can be constructed as an integral unit together with mouthpiece portion 202, extender portion 204, or both mouthpiece portion 202 and extender portion 204. Outlet port 228 of sensing chamber portion 206 can be configured to contain air flow restrictor 212. Air flow restrictor 212 can be designed to allow exhaled breath to exit through outlet port 228 in a manner that is restricted as compared to an outlet port lacking an air flow restrictor. In some cases, air flow restrictors 208, 210, and 228 can be configured such that the air flow rate through sensing chamber portion 206 is between 0.5 and 0.01 L/second (e.g., about 0.05 L/second).

Sensing chamber portion 206 can contain opening 230. Opening 230 can be configured such that probe 214 can be positioned to sense NO within sensing chamber 206. Any type of probe (e.g., amperometric probe) can be used provided that it is capable of sensing NO. Examples of probes that can be used include, without limitation, those produced or sold by Diamond General (Ann Arbor, Mich.), World Precision Instruments (Sarasota, Fla.), Innovative Instruments, Inc. (Tampa, Fla.), Inter Medical Co., Ltd. (Nagoya, Japan), and TSI Incorporated (Shoreview, Minn.). In some cases, a selective amperometric combination electrode or differential electrode can be used as a probe to sense NO as described herein. In some cases, a non-chemiluminescence, amperometric probe capable of sensing NO can be used as described herein. For example, an electrochemical probe capable of sensing NO can be used as described herein. In some cases, probe 214 can be designed to sense pH, moisture, and temperature within a sensing chamber.

Probe 241 can be wired via wire 232 to an analyzer capable of receiving NO sensing data from the probe. Such an analyzer also can provide output about NO levels detected within a sensing chamber. Examples of analyzers include, without limitation, those produced or sold by ESA Biosciences, Inc. (Chelmsford, Mass.), Innovative Instruments, Inc. (Tampa, Fla.), Inter Medical Co., Ltd. (Nagoya, Japan), Diamond General (Ann Arbor, Mich.), TSI Incorporated (Shoreview, Minn.), EDAQ (New South Wales, Australia), World Precision Instruments (Sarasota, Fla.). In some cases, a WPI Apollo 4000 analyzer, a DUO18 analyzer, or ESA Biostat can be used. In some cases, probe 214 can be wireless such that NO sensing data is sent from probe 214 to an analyzer in a wireless manner.

NO sensing device 200 can be constructed so that material within the device can be moistened with, for example, water, a saline solution, or a water gel composite prior to sensing NO within sensing chamber portion 206. For example, a filter designed to moisten exhaled breath can be incorporated into device 200. Such a filter can be located anywhere within device 200. For example, a filter designed to moisten exhaled breath can be located within mouthpiece portion 202, within extender portion 204, or within sensing chamber portion 206. In some cases, an NO sensing device provided herein can contain a fluid reservoir capable of holding fluid such as water. Such a fluid reservoir can be configured to deliver fluid to a filter designed to moisten exhaled breath. For example, a fluid reservoir can be configured to deliver a fine mist to the inner surface of mouthpiece portion 202, extender portion 204, sensing chamber portion 206, a filter within mouthpiece portion 202, extender portion 204, or sensing chamber portion 206, a flow restrictor within mouthpiece portion 202, extender portion 204, or sensing chamber portion 206, or a combination thereof. Any type of dispensing unit can be used to deliver fluid. For example, a push bulb spray unit can be actuated by a user to deliver a fine mist to a filter located within mouthpiece portion 202. In some cases, a user can use a spray bottle to moisten an inner surface of device 200.

In a manner of use, device 200 can be held by a human user such that the human user can exhale breath into mouthpiece portion 202 or can be inserted into the mouth or nostril of an animal user. In some cases, an inner surface of device 200 can be moistened with water via a spray bottle before being used by the user. Once the NO sensing device is in position, the user can breath normally or under various conditions (e.g., while walking or running on a tread-mill, while mediating (e.g., relaxing), or while sleeping) for a pre-selected time period (e.g., 0.5, 1, 2, 5, 10, 20, or 30 minutes). In some cases, NO can be measured in users having a particular disease or condition. For example, NO can be measured in a group of asthma patients. The probe can be used to sense NO within the sensing chamber portion in either a continuous mode or at pre-set intervals (e.g., once every 10, 30, or 60 seconds). Prior to making a NO measurement, the probe can be calibrated. For example, when sensing exhaled NO, an initial three point calibration can be performed followed by daily two point calibrations (e.g., zero and a point in the expected range). See, e.g., An official statement of the American Thoracic Society adopted by the ATS Board of Directors, July 1999 (Am. J. Respir. Crit. Care Med., 160(6):2104-17 (1999)). In some cases, device 200 can be configured to measure NO in a manner that is independent of flow rate. In some cases, the flow rate within sensing chamber 200 during calibration can match the flow rate obtained during use by a user. Such a flow rate can be between 0.5 and 0.01 L/second (e.g., about 0.05 L/second).

The invention will be further described in the following examples, which do not limit the scope of the invention described in the claims.

### EXAMPLES

### Example 1

### NO Gas Calibration

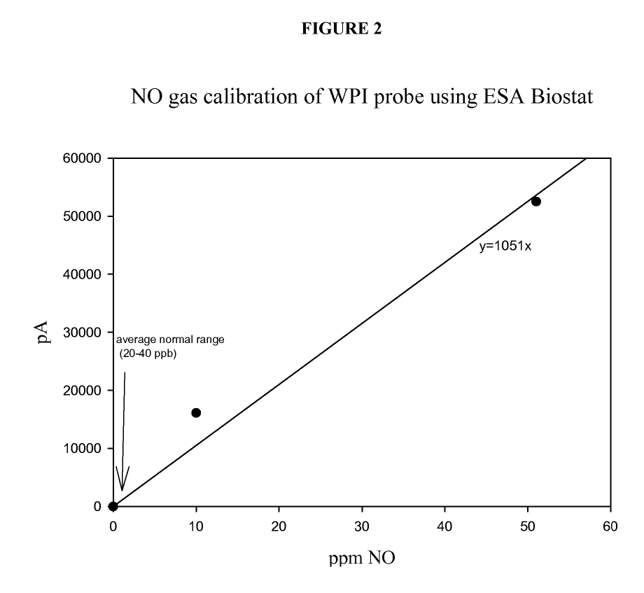

NO gas in O₂-free N₂ was obtained from Scott Specialty Gases. Cylinders containing 58 L of gas (Scotty Transportables) were connected to a model 38 single stage gas regulator (Scott Specialty Gases). The gas was used to fill a latex balloon in a water saturated atmosphere. The NO electrode was inserted into the balloon in a manner that formed a seal not allowing the gas to escape until vented manually. The balloon was filled from a tube connected to the regulator. The current was recorded using an ESA Biostat (ESA, Ma) connected to a WPI 100 um Flexible NO probe (World Precision Instruments, Sarasota, Fla.). Calibration gases included 0, 10, and 51 ppm NO. Calibration yielded a curve with 1051 pA for every ppm NO (FIG. 2; R²=0.9776). Normal exhaled NO has been shown to be about 20-40 ppb (Haight et al., Lung, 184(2):113-9 (2006)). Based on the sensitivity of the detector, this system can detect as low as 1 ppb NO. The detector system can resolve amperage differences of 0.5 pA. A 1 pA change is greater than the noise of the system, and 1 pA is equal to about 1 ppb.

### Example 2

### Sensing NO in Exhaled Breath

People were allowed to sit and relax for 20 minutes after having been active. Using the mask designed for exhaled NO measurements, an average NO value of 28±10 ppb (n=5) was detected. When the mask was dry, no NO was detected. When moisture was added directly to the mask, however, NO was detected. The moisture can aid in delivering NO, which is normally in a humid physiological environment, to the membrane and electric circuit of the probe.

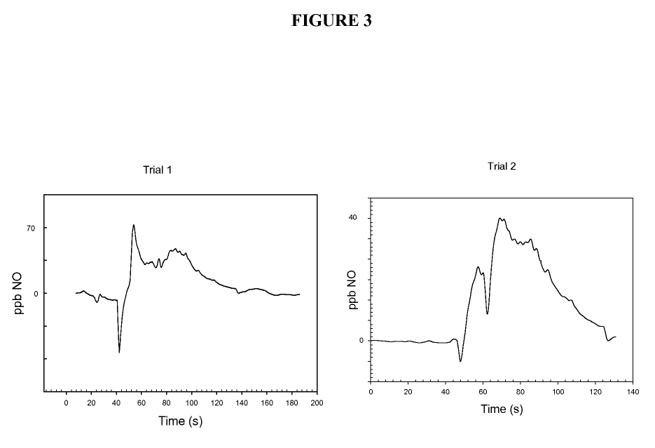

NO measurements also were obtained from people who sat without a relaxation period. The mean NO value per exhaled breath in 10 people was found to be 51±14 ppb NO (FIG. 3). In each case, the NO value was determined by the plateau of the peak for 10 seconds as described elsewhere (An official statement of the American Thoracic Society adopted by the ATS Board of Directors, July 1999 (Am. J. Respir. Crit. Care Med., 160(6):2104-17 (1999)).

### Example 3

### Sensing NO in Ambient Air and Air from a CO₂ tank

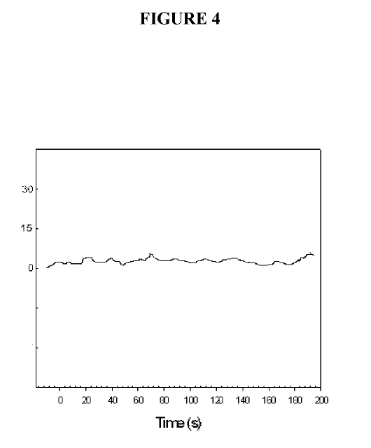

An NO probe was used to measure NO in ambient air at 25° C. The probe detected NO at a level of 1±1 ppb (FIG. 4). These results demonstrate that room air contributes a low level of noise to the detection system, which does not interfere with the NO reading.

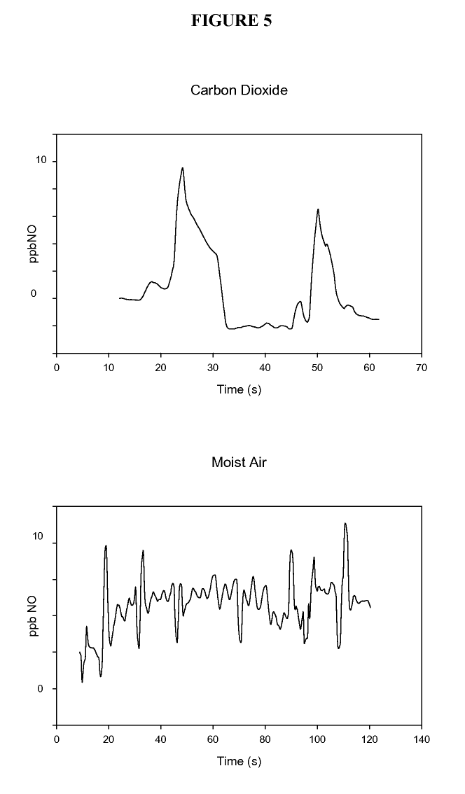

An NO probe was used to measure NO in air obtained from a tank reported to contain 100% CO₂. The probe detected NO at a level of 6.5±2.5 ppb, clearly well below that of NO (FIG. 5, top). These results demonstrate that 100% CO₂ slightly affects the detection system. Exhaled breath contains less than 5% CO₂. When humidified air was passed over the detector, the level of NO measured was 3±2 ppb (FIG. 5, bottom). These results demonstrate that humidified air contributes to the noise in the detection system but at a level that does not interfere with the actual NO level.

### Example 4

### Sensing NO in Exhaled Breath after Exercise

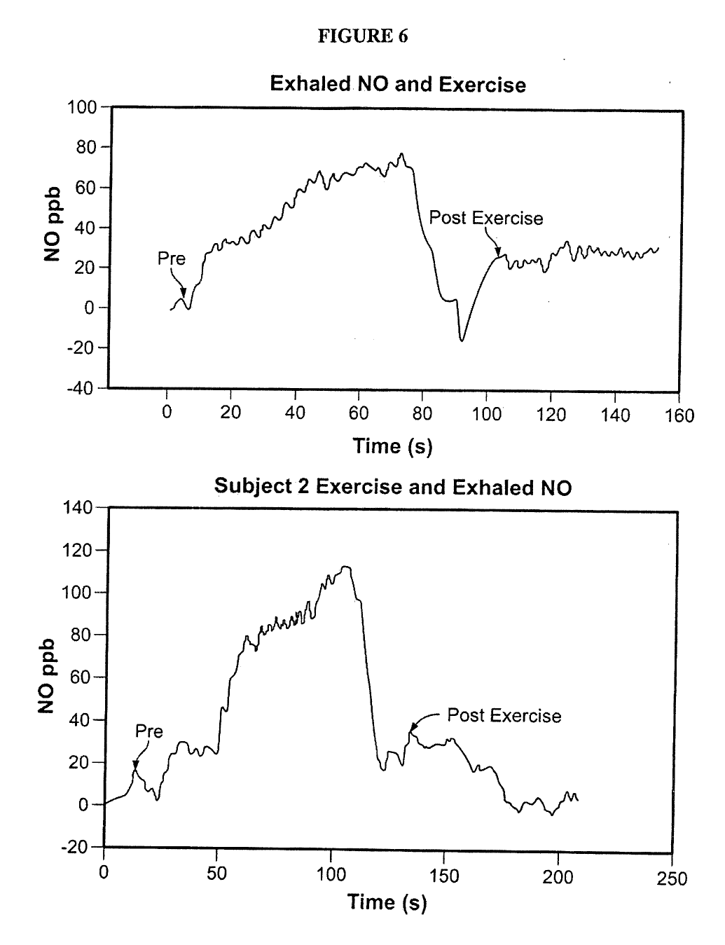

Control NO values were measured from ten sitting humans two minutes after they were walking. After these measurements, each human ran about 180 yards and immediately thereafter sat down for another NO reading. The control NO levels (just after sitting) were 98.54±36 ppb, while after running the NO levels were 26.5±12 ppb. FIG. 6 contains representative readings for two humans.

### Example 5

### Sensing NO in Exhaled Breath after Relaxing

Human subjects were brought into the lab and immediately asked to sit and place the mask over their mouth and nose. NO was measured after an immediate plateau of NO was noted on the meter (after about 60 seconds). Subjects (5) were then asked to sit quietly (relax, close their eyes) for 10 minutes. The NO mask was replaced onto the subjects, and peak NO was recorded again (about 60 seconds). Peak heights were 32±5 ppb initially and 72±19 ppb after 10 minutes of relaxation. This confirms earlier observations that relaxation increases NO in exhaled air (Stefano et al., Brain Research: Brain Research Reviews, 35:1-19 (2001); Dusek et al., Med. Sci. Monit., 12:CR1-10 (2006); and Stefano et al., Pharmacol. Res., 43:199-203 (2001)) and that the devices provided herein can measure NO.

### Example 6

### Sensing NO in Exhaled Breath from Dogs

Three dogs were used in this study. Briefly, an NO mask was placed over the nose and mouth of each dog, and peak NO levels were recorded (about 30 seconds). The average peak NO levels were 14.5±2.4 ppb NO. This confirms that NO can be accurately measured in exhaled air from animals such as dogs.

### Example 7

### Sensing NO in Exhaled Breath from Cattle

28 cattle (adult cows and bulls) were used in this study. Briefly, a tubular NO sensing device was placed into a nostril of each cow or bull, and peak NO levels were recorded (about 60 seconds). The average peak NO levels were 18.7±1.9 ppb NO. This confirms that NO can be accurately measured in exhaled air from animals such as cows and bulls.

### Example 8

### NO Gas Calibration

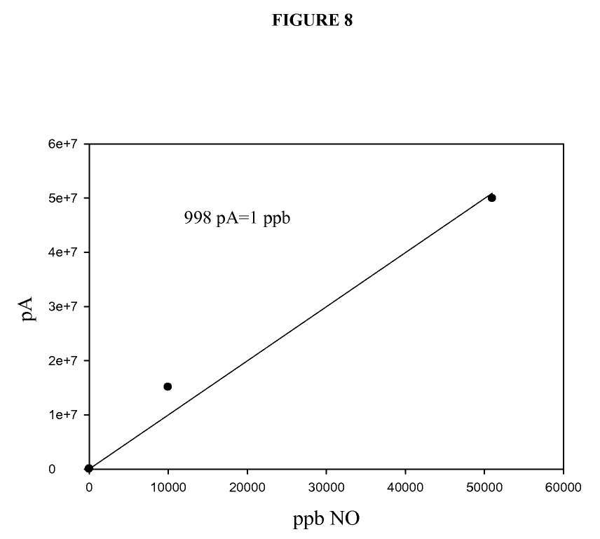

NO gas in O₂-free N₂ was obtained from Scott Specialty Gases. For the 0, 10, and 51 ppm standards, cylinders containing 58 L of gas (Scotty Transportables) were connected to a model 38 single stage gas regulator (Scott Specialty Gases). The 52 ppb standard was also obtained from Scott Specialty gases. The gas was received in a mixture with O₂-free N₂ at 2000 PSI and was regulated with a stainless steel CGA 660 regulator (General Welding, Westbury, N.Y.). The gas was connected to a plastic tube that directed the flow past a probe inserted through the top of a T-shaped connector (see, e.g., FIG. 7). The flow rate used was 3 L/minute as measured using a minimaster flow meter model MMA-22 (Dwyer Instruments, Inc., Michigan City, Ind.). The NO electrode was inserted into the T-shaped connector in a manner that formed a seal not allowing the gas to escape until it passed the probe. The current was recorded using an ESA Biostat (ESA, Ma) connected to a 700 μm flexible NO probe (Innovative Instruments, Tampa, Fla.). Calibration gases included 0, 52 ppb, 10 ppm, and 51 ppm NO, and yielded results of 0 pA, 44,668 pA, 15,119,000 pA, and 49,928,000 pA, respectively. Calibration yielded a curve with 998 pA for every ppb NO (FIG. 8; R²=0.9836). Normal exhaled NO has been shown to be about 20-40 ppb (Haight et al., Lung, 184(2):113-9 (2006)). Based on the sensitivity of the detector, this system can detect lower than 1 ppb NO.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A device for sensing nitric oxide in exhaled breath, wherein said device comprises a mask portion configured to cover the nose and mouth of a mammal, thereby forming a sensing chamber when applied to said mammal, and a nitric oxide probe for sensing nitric oxide within said sensing chamber.

2. The device of claim 1, wherein said mask portion comprises cloth or paper.

3. The device of claim 1, wherein said mammal is a human.

4. The device of claim 1, wherein said device comprises a connector for positioning said mask portion over the nose and mouth of a mammal.

5. The device of claim 4, wherein said connector comprises an elastic cord.

6. The device of claim 1, wherein said mask portion defines an opening for said nitric oxide probe.

7. The device of claim 1, wherein said mask portion comprises moisture.

8. The device of claim 1, wherein said device comprises a fluid reservoir capable of providing moisture to a surface of said mask portion.

9. The device of claim 1, wherein said surface is an inner surface of said mask portion.

10. The device of claim 1, wherein said mask portion comprises a pleat.

11. A method for making a device for sensing nitric oxide in exhaled breath, wherein said method comprises:
(a) obtaining a nitric oxide probe for sensing nitric oxide;
(b) obtaining a mask portion configured to cover the nose and mouth of a mammal, thereby forming a sensing chamber when applied to said mammal, and
(c) adding said probe to said mask portion such that said probe is capable of sensing NO within said sensing chamber.

12. The method of claim 11, wherein said mask portion comprises cloth or paper.

13. The method of claim 11, wherein said mammal is a human.

14. The method of claim 11, wherein method comprises adding, to said mask portion, a connector for positioning said mask portion over the nose and mouth of a mammal.

15. The method of claim 14, wherein said connector comprises an elastic cord.

16. The method of claim 11, wherein said mask portion defines an opening for said nitric oxide probe.

17. The method of claim 11, wherein said mask portion comprises moisture.

18. The method of claim 11, wherein said method comprises adding, to said mask portion, a fluid reservoir capable of providing moisture to a surface of said mask portion.

19. The method of claim 18, wherein said surface is an inner surface of said mask portion.

20. The method of claim 11, wherein said mask portion comprises a pleat.

21. A method for sensing nitric oxide in exhaled breath, wherein said method comprises:
(a) obtaining a device comprises a mask portion configured to cover the nose and mouth of a mammal, thereby forming a sensing chamber when applied to said mammal, and a nitric oxide probe for sensing nitric oxide within said sensing chamber;
(b) applying said device to the face of said mammal, thereby forming said sensing chamber; and
(c) sensing exhaled NO within said sensing chamber via said probe.

22. The method of claim 21, wherein said mask portion comprises cloth or paper.

23. The method of claim 21, wherein said mammal is a human.

24. The method of claim 21, wherein said device comprises a connector for positioning said mask portion over the nose and mouth of a mammal.

25. The method of claim 24, wherein said connector comprises an elastic cord.

26. The method of claim 21, wherein said mask portion defines an opening for said nitric oxide probe.

27. The method of claim 21, wherein said mask portion comprises moisture.

28. The method of claim 21, wherein said device comprises a fluid reservoir capable of providing moisture to a surface of said mask portion.

29. The method of claim 28, wherein said surface is an inner surface of said mask portion.

30. The method of claim 21, wherein said mask portion comprises a pleat.

31. The method of claim 21, wherein said method comprises applying moisture to a surface of said mask portion before or after said applying step (b).

32. A device for sensing nitric oxide in exhaled breath of a mammal, wherein said device comprises a mouthpiece portion, an extender portion, and a nitric oxide sensing chamber portion configured to allow exhaled breath to travel from said mouthpiece portion to said nitric oxide sensing chamber portion by traveling through said extender portion, wherein said device comprises a flow restrictor within said mouthpiece portion, said extender portion, or said nitric oxide sensing chamber portion, and wherein said device comprises a nitric oxide probe for sensing nitric oxide within said nitric oxide sensing chamber portion.

33. The device of claim 32, wherein said mammal is a human.

34. The device of claim 32, wherein said device comprises moisture.

34. The device of claim 32, wherein said device comprises a fluid reservoir capable of providing moisture to a inner surface of said device.

35. A device for sensing nitric oxide in exhaled breath, wherein said device comprises a portion configured to cover the nose or mouth of a mammal, thereby forming a sensing chamber when applied to said mammal, and an amperometric, non-chemiluminescence probe for sensing nitric oxide within said sensing chamber.

36. The device of claim 35, wherein said portion is a mask portion comprising cloth or paper.

37. The device of claim 35, wherein said mammal is a human.

38. The device of claim 35, wherein said device comprises a connector for positioning said portion over the nose and mouth of a mammal.

39. The device of claim 38, wherein said connector comprises an elastic cord.

40. A method for sensing nitric oxide in exhaled breath, wherein said method comprises:
(a) obtaining a device comprises a portion configured to cover the nose or mouth of a mammal, thereby forming a sensing chamber when applied to said mammal, and an amperometric, non-chemiluminescence probe for sensing nitric oxide within said sensing chamber;
(b) applying said device to said mammal, thereby forming said sensing chamber; and
(c) sensing exhaled NO within said sensing chamber via said probe.

41. The method of claim 40, wherein said portion comprises a mask portion comprising cloth or paper.

42. The method of claim 40, wherein said mammal is a human.

43. The method of claim 40, wherein said method comprises applying moisture to a surface of said device.

44. The method of claim 40, wherein said device comprises a mouthpiece portion, an extender portion, and a nitric oxide sensing chamber portion configured to allow exhaled breath to travel from said mouthpiece portion to said nitric oxide sensing chamber portion by traveling through said extender portion, wherein said device comprises a flow restrictor within said mouthpiece portion, said extender portion, or said nitric oxide sensing chamber portion.
